(19) United States
(12) Patent Application Publication
(10) Pub. No.: US 2014/0330277 A1
(43) Pub. Date: Nov. 6, 2014

(54) SURGICAL TARGETING GUIDE

(71) Applicants: Peter J. Ogrodnik, Stoke-on-Trent (GB); Peter B.M. Thomas, Betley (GB)

(72) Inventors: Peter J. Ogrodnik, Stoke-on-Trent (GB); Peter B.M. Thomas, Betley (GB)

(73) Assignee: INTELLIGENT ORTHOPAEDICS LIMITED, Stoke on Trent, Staffordshire (GB)

(21) Appl. No.: 14/236,059

(22) PCT Filed: Jul. 20, 2012
(86) PCT No.: PCT/GB2012/051735

§ 371 Date: Jul. 2, 2014

(30) Foreign Application Priority Data
Jul. 29, 2011 (GB) 1113064.8

(51) Int. Cl. A61B 17/17

(52) U.S. Cl. CPC A61B 17/17
USPC 606/87

(57) ABSTRACT

A targeting guide for locating a surgical target site, for example a hole in a distal end of an implanted intramedullary nail, the targeting guide comprising: a first indicator having an identifiable reference point, the first indicator being mounted on a first handle having a first longitudinal axis; a second indicator having an identifiable reference point, the second indicator being mounted on a second handle having a second longitudinal axis; the indicators being radially spaced from one another with respect to said target site and being at least partially radiopaque so that they are imageable such that their respective reference points can be identified; a rig on which the first and second indicators are mountable such that each indicator is independently moveable with respect to the rig along the respective longitudinal axis of its handle, independently moveable with respect to the rig along at least one further axis and lockable with respect to the rig in a selected position along each of said axes; wherein, in use, the reference point of each indicator is capable of being aligned with a reference point of the target site using imaging means, the aligned indicators providing a guide to the target site for a drill, trocar or other surgical instrument or tool.

This invention relates to apparatus and methods for targeting or locating a hole in a plate, nail, or other surgical device that has already been implanted in the body, or any other hole or aperture that appears visible using x-ray or other imaging means, for example to facilitate the accurate insertion drilling through the hole for internal fracture fixation.

### BACKGROUND

Several techniques are well-known for the holding together of fractured parts of a bone while healing takes place. The fractured parts may be externally fixated, using a fixator of the type described in GB2427171B for example. Alternatively plates or intramedullary nails may be implanted.

Before such fixation can take place, the fracture must be reduced so that the bone fragments are in the correct position for fixation and healing. A well-known fracture reduction device is described in EP0984729B and marketed under the trade mark STORM®.

In both fracture reduction and fixation techniques, there is often the need to accurately locate holes that are not visible to the eye. This is particularly an issue at the distal end of an intramedullary nail, for example, where the distal end will be located some distance from the opening of the surgical site. Conventionally this is done with the assistance of X-ray location (the nail hole appearing as a light circle on the X-ray image of the radiopaque nail) or using a targeting arm unique to the nail, but this can be problematic.

Once implanted, but not yet fixed to the bone, the distal end of the nail can move with respect to the bone. In addition, the skin can move with respect to both the nail and the bone, such that measurement external to the skin is not sufficiently accurate. Trial and error can be used which may undesirably result in several drill holes before the correct location is found.

US-A-2004/0106922 (Snyder) describes a method and apparatus for installing a number of distal screws into an intramedullary nail implanted in a patient. The apparatus includes a foundation which is fixedly coupled to the patient, a targeting insert having a targeting spike, a guiding insert for guiding an installation tool used in installing a distal screw, a targeting/guiding device coupled to the foundation and adaptable for having the targeting insert and the guiding insert coupled thereto, and a handle removably coupled to the targeting/guiding device. When the targeting insert is coupled to the targeting/guiding device, the targeting/guiding device may be movable by an operator or surgeon with the use of the handle so as to align the targeting spike with a respective distal screw hole in the intramedullary nail. Afterwards, the targeting/guiding device may be locked in place by the operator or surgeon with the use of the handle, the targeting insert removed, and the guiding insert coupled to the targeting/guiding device. Successful operation of this device requires the whole apparatus to first be attached to the patient's body by a strap and/or fixation pins. A locking targeting guide is then manipulated to achieve alignment. However, this device requires the targeting spike to be located centrally and any mis-angulation makes accurate alignment extremely difficult to achieve.

US2004/0167533 describes a radiolucent aiming guide for locating and drilling through the holes in the distal end of an implanted intramedullary nail. The aiming guide permits a surgeon to accurately position a trocar or drill over a nail hole in order to make an incision and hole for insertion of a locking screw. A radiolucent handle is provided, to which is perpendicularly connected a substantially radiolucent protection sleeve containing a trocar, the protection sleeve and trocar each having a radiopaque tip which cast concentric circular images on an X-ray monitor. When the aiming guide is correctly positioned, the concentric circular images are superimposed over the circular image of the nail hole it is desired to locate. Then the trocar can be replaced by a drill sleeve through which drilling can take place. Successful operation of this aiming guide is dependent upon correctly aligning both the hidden hole and the aiming guide with the X-ray monitor and rigidly holding both the handle and protection sleeve mutually perpendicularly with respect to the hidden hole.

It is an object of the present invention to provide apparatus and methods for targeting a surgical site that seeks to improve upon the prior art techniques.

### BRIEF SUMMARY OF THE DISCLOSURE

The present invention is defined in the appended claims.

In accordance with a first aspect of the present invention there is provided a targeting guide for locating a surgical target site, for example a hole in a distal end of an implanted intramedullary nail, the targeting guide comprising:
a first indicator having an identifiable reference point, the first indicator being mounted on a first handle having a first longitudinal axis;
a second indicator having an identifiable reference point, the second indicator being mounted on a second handle having a second longitudinal axis;
the indicators being radially spaced from one another with respect to said target site and being at least partially radiopaque so that they are imageable such that their respective reference points can be identified;
a rig on which the first and second indicators are mountable such that each indicator is independently moveable with respect to the rig along the respective longitudinal axis of its handle, independently moveable with respect to the rig along at least one further axis and lockable with respect to the rig in a selected position along each of said axes;
   wherein, in use, the reference point of each indicator is capable of being aligned with a reference point of the target site using imaging means, the aligned indicators providing a guide to the target site for a drill, trocar or other surgical instrument or tool.

Preferably, the identifiable reference point of said first indicator and/or said second indicator and/or said target site is a centre point.

In a preferable embodiment, said rig comprises an arc portion and movement along said at least one further axis comprises movement along said arc portion.

Movement along said at least one further axis may comprise a pivoting movement. Alternatively or additionally, said handles are radiolucent.

In a preferable embodiment, said first indicator is a ring, wherein the ring preferably has one or more alignment markers thereon to facilitate location of said identifiable reference point. In a particularly preferable embodiment, at least one of said alignment markers is a pointer extending radially with respect to the centre point of said ring. At least one of said alignment markers may be a radiolucent hole through a peripheral surface of said ring. The first indicator and second indicators may be the same shape. The rig portion may include fixings for attachment of the rig portion to a fracture reduction machine, table, frame or other supporting structure. In any embodiment, the rig portion may comprise a two-part rig portion, the first indicator being mountable on a first part of the rig portion and the second indicator being mountable on a second part of the rig portion.

Preferably, the rig portion comprises at least one mounting block for at least one of said first and second indicators. Further preferably, the rig portion comprises first and second mounting blocks for said first and second indicators respectively. Additionally or alternatively, the or each mounting block is moveable with respect to said rig portion, for example by running the mounting block along a track on said rig portion. In any embodiment, said movement of said first and second indicators can be finely adjusted using controls on said mounting blocks.

The rig may further comprise a locking mechanism for independently selectively locking each of said first and second indicators in a desired position. The first and/or said second indicators may include a generally spherical bearing therein, a reference point of said bearing being generally aligned with the reference point of the indicator, the bearing being able to articulate with respect to the indicator and the bearing having a hole therethrough for receipt of a drill, trocar or other surgical instrument or tool. The spherical bearings may comprise plastics or any other radiolucent material.

In one preferable embodiment, the indicators comprise metal. In a further or alternative embodiment, the first indicator is radially nearer said target site than said second indicator.

Preferably, the indicators are at least radiopaque so that they cast an image on an X-ray monitor from which their respective centre points can be identified, and wherein the imaging means comprise an X-ray monitor.

In one preferable embodiment, the targeting guide further comprises a computerised or robotic control system for moving the first and second indicators with respect to the rig.

In accordance with a second aspect of the present invention, there is provided a method of locating a surgical target site using a targeting guide according to the first aspect of the present invention, comprising the steps of:
mounting the rig of said targeting guide in the vicinity of said target site;
aligning an imaging means with the reference point of the target site;
moving said first indicator along said first longitudinal axis and/or along said further axis until its reference point is aligned with the reference point of the target site;
locking said first indicator with respect to said rig;
moving said second indicator along said second longitudinal axis and/or along said further axis until its reference point is aligned with the reference point of the target site;
locking said second indicator with respect to said rig.

In one preferable embodiment, a computerised or robotic control system is used to at least partially move the first and second indicators with respect to the rig.

### BRIEF DESCRIPTION OF THE DRAWINGS

Embodiments of the invention are further described hereinafter with reference to the accompanying drawings, in which:

FIG. 1 is a schematic view of an intramedullary nail with hidden hole, in order to illustrate a principle of the present invention;

FIG. 2 shows the intramedullary nail of FIG. 1 with two indicators aligned with the hole;

FIGS. 3 and 4 show the intramedullary nail aligned with two indicators enabling a drill to be accurately placed into the hole;

FIGS. 5A, 5B and 5C are further views, drawn to a larger scale, showing the indicators approaching one another, almost aligned, and actually aligned with the hole respectively;

FIG. 6 shows one of the indicators in more detail;

FIG. 7 shows both of the indicators in more detail;

FIGS. 8-10 are perspective views showing the indicators with optional spherical balls therein;

FIGS. 11-14 show the indicators mounted on a rig;

FIG. 15 is a schematic view of the hole in the intramedullary nail, as viewed by the radiographer, with one indicator and the arc portion of the rig also visible;

FIG. 16 shows the centre point of the indicator aligned with the centre point of the hole in the intramedullary nail;

FIG. 17 shows a drill guided into the hole in the intramedullary nail by the aligned indicators;

FIGS. 18 and 19 show example applications of the invention other than intramedullary nails;

FIGS. 20 and 21 show an alternative embodiment in which pivotable arms are provided instead of an arc portion;

FIG. 22A shows an indicator with an alignment plug in place;

FIG. 22B is a perspective view of the indicator of FIG. 22A, showing the cross-hairs of the alignment plug;

FIG. 22C is a perspective view of an alignment plug having radiopaque cross-hairs; and

FIG. 22D is a perspective view of an alternative alignment plug having a radiopaque ball instead of cross hairs.

### DETAILED DESCRIPTION

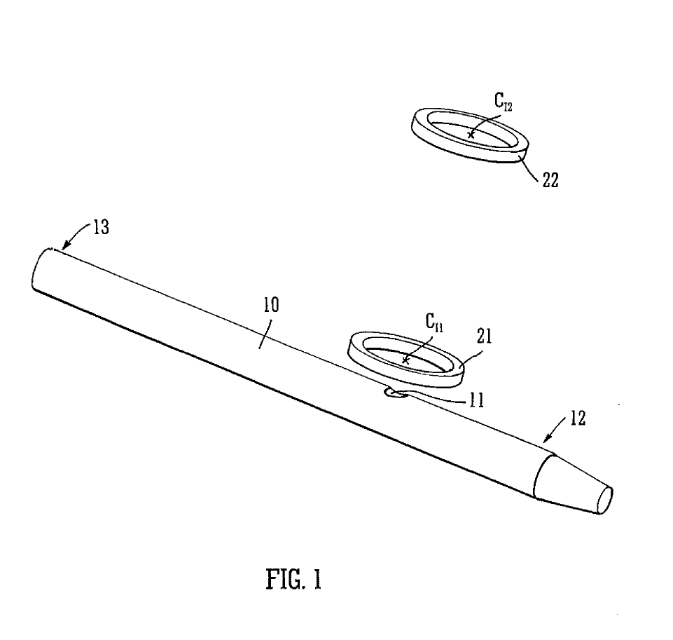

FIG. 1 is a perspective schematic view of an intramedullary nail (“IM”) 10, having a hole 11 near the distal end 12 thereof which it is desired to accurately locate. Once the IM is implanted, most of the IM (including the hole 11 and distal end 12) is no longer visible as it is in the body under the skin and can only be viewed using radiography techniques.

The proximal end 13 of the IM may still be visible at the surgical site. However, it is not possible to accurately determine where the hole 11 is located simply based on the location of the proximal end 13 and known dimensions of the IM 10 because the IM could deform, slip and/or rotate during implantation.

The present invention is based upon the principle of aligning two independently moveable indicators 21, 22, each having a known reference point, with a reference point at the hole 11 using X-ray or other known imaging techniques. Preferably, these reference points are centre points but they need not be mathematically exact centres and other reference points may be used so long as their position can be reliably and absolutely determined. In the exemplary embodiment described below, the reference points are centre points. In the described embodiment, the indicators 21, 22 are generally circular but they may be ellipses or any other geometric shapes having definable centre points. The indicators are at least partially radiopaque in order for their centres to be apparent, with respect to the hole 11.

It is preferable for the indicators 21, 22 to be spaced from one another as much as possible in the radial direction from the IM in order to maximise accuracy. It is also preferable for the indicator 21 nearest the hole 11 to be located as close as possible to the hole (in practice this means as close to the skin as possible).

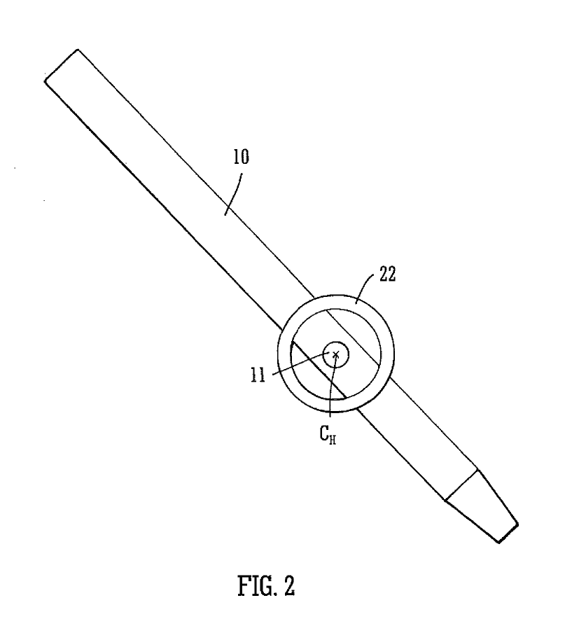

Alignment is defined with respect to the centre of the hole 11, indicated as C_{H} in FIG. 2. It is well-known for a radiographer to be able to align an X-ray device with C_{H}, giving a view of the IM and hole as that illustrated in FIG. 2. The present invention is concerned with the alignment of the moveable indicators 21, 22 so that their respective centre points C_{I1}, C_{I2} are aligned with C_{H}, as in FIG. 2.

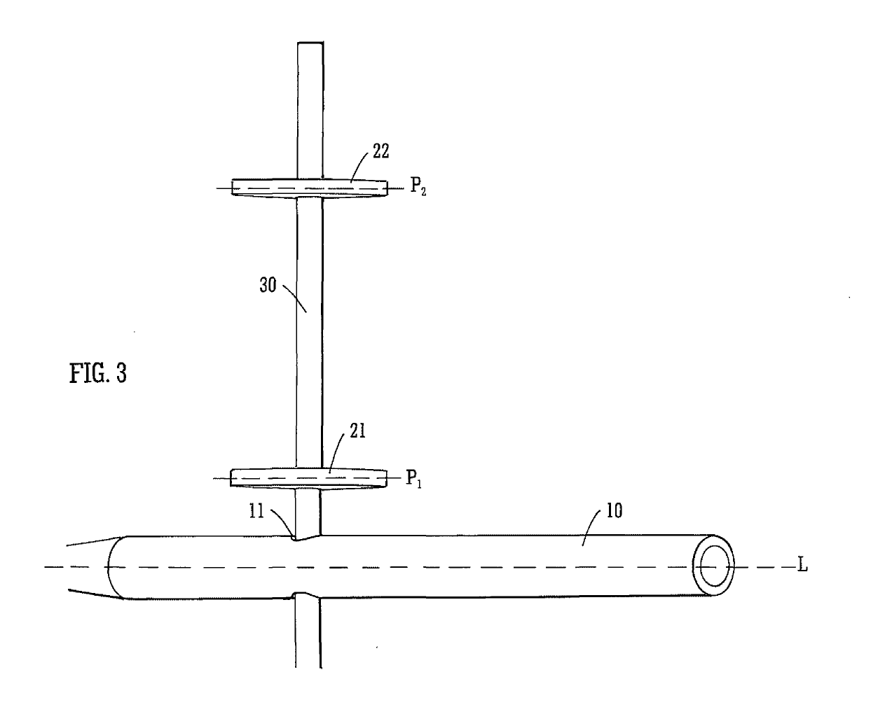

Once the centre point C_{H} of hole 11 is aligned with each of the centre points C_{I1}, C_{I2} of the indicators 21, 22 respectively, a drill 30 can be accurately inserted through the hole 11 using the indicators as guides, as illustrated in FIG. 3.

FIG. 3 shows a situation in which the longitudinal axis L of the IM is substantially parallel with the planes in which each of the indicators 21, 22 are located.

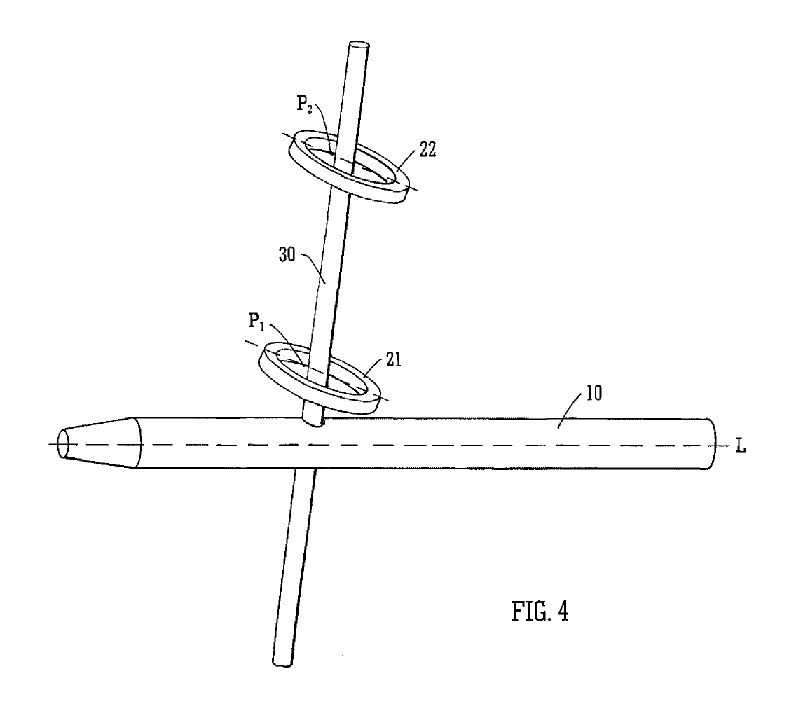

However, as shown in FIG. 4, even if the longitudinal axis of the IM 11 is not parallel with the plane of one or both indicators 21, 22, then alignment is still possible using the centre points C_{I1}, C_{I2} of the indicators. In such a case, the circular indicators will appear as ellipses when viewed by the X-ray device, but since ellipses have definable centre points, alignment with the hole is still possible.

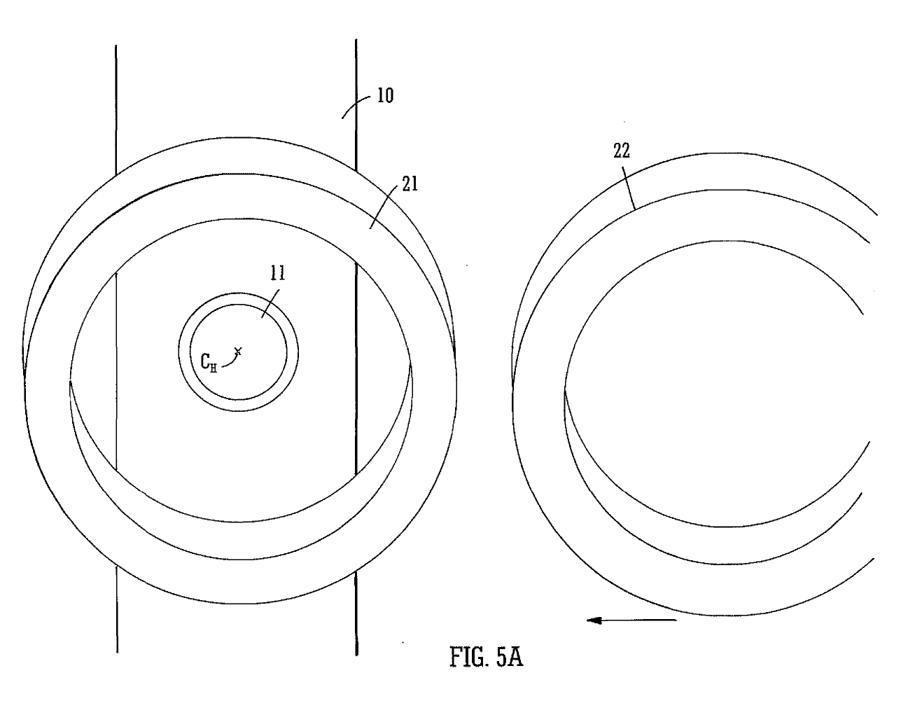

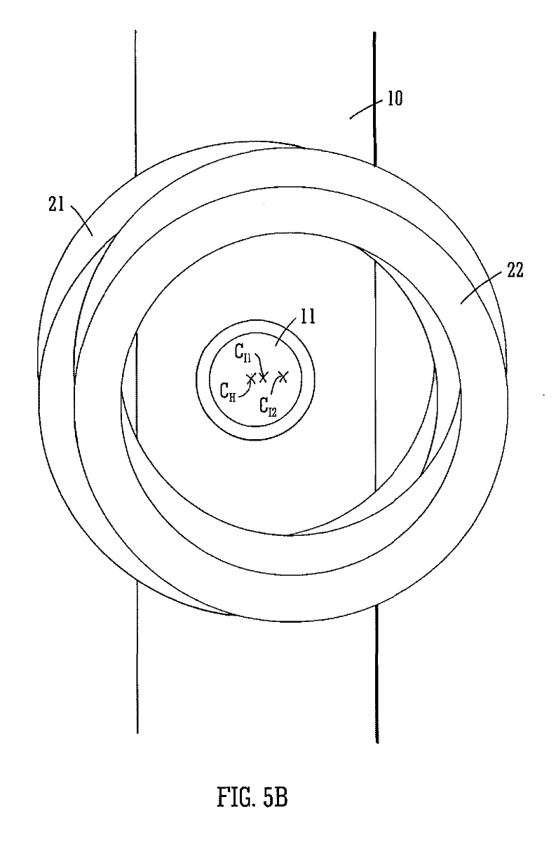

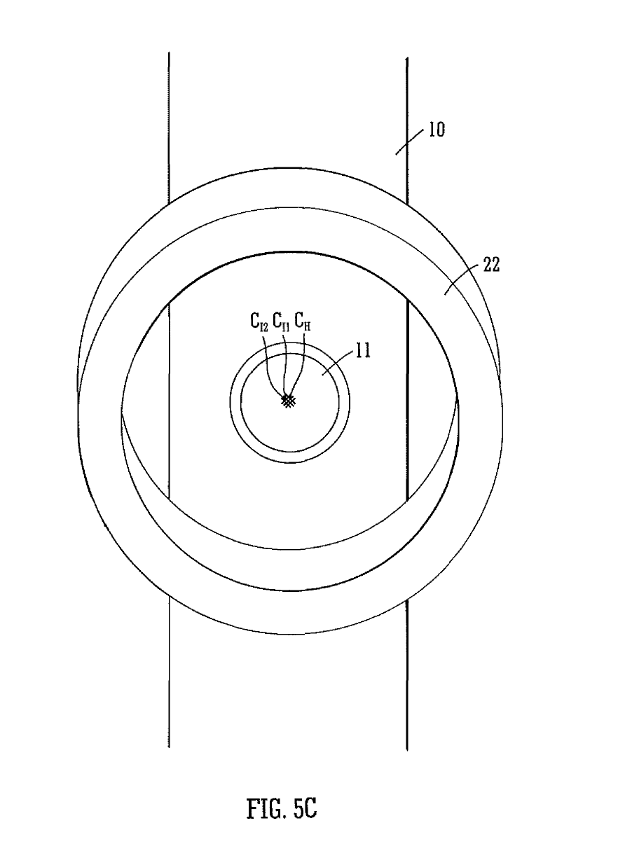

FIG. 5A shows schematically how the moveable indicator 22 approaches the indicator 21 (in the direction indicated by the arrow). In FIG. 5B, the centre points of indicators 21, 22 are almost aligned with one another and with the centre point of the hole C_{H}. In FIG. 5C, the centre points of the indicators 21, 22 are properly aligned with the centre point of the hole C_{H}.

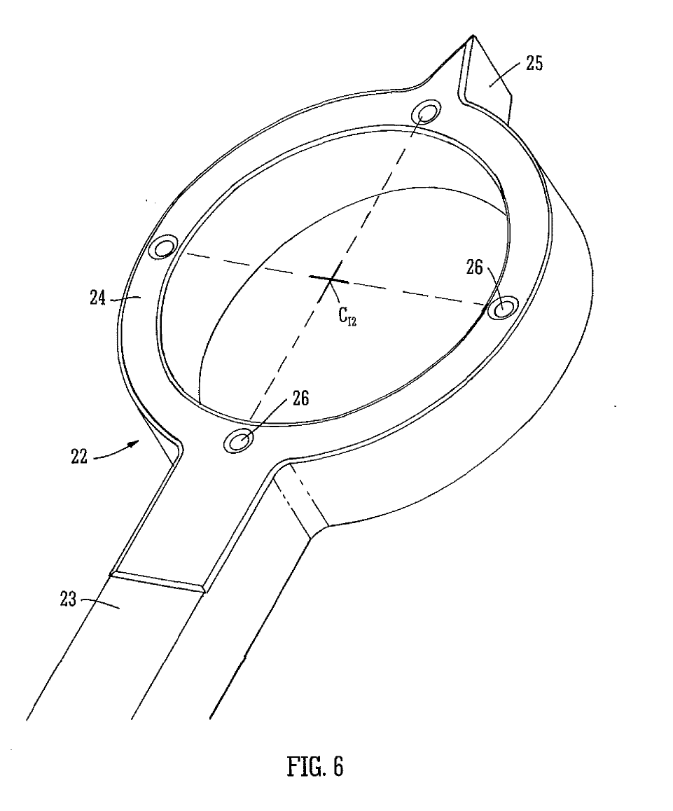

In one embodiment, as shown in FIG. 6, the indicator 22 comprises a radiolucent handle 23 attached to a radiopaque ring 24. The ring 24 has a pointer 25 to facilitate alignment with the hidden hole 11 (not shown in FIG. 6) and may also include further alignment markers or holes 26 to help define the centre point C_{I2} of the ring 24.

FIG. 7 shows both of the indicators 21, 22 with their respective centre points C_{I1}, C_{I2}. The indicators 21, 22 are independently moveable with respect to one another in directions set out by way of example in further detail below.

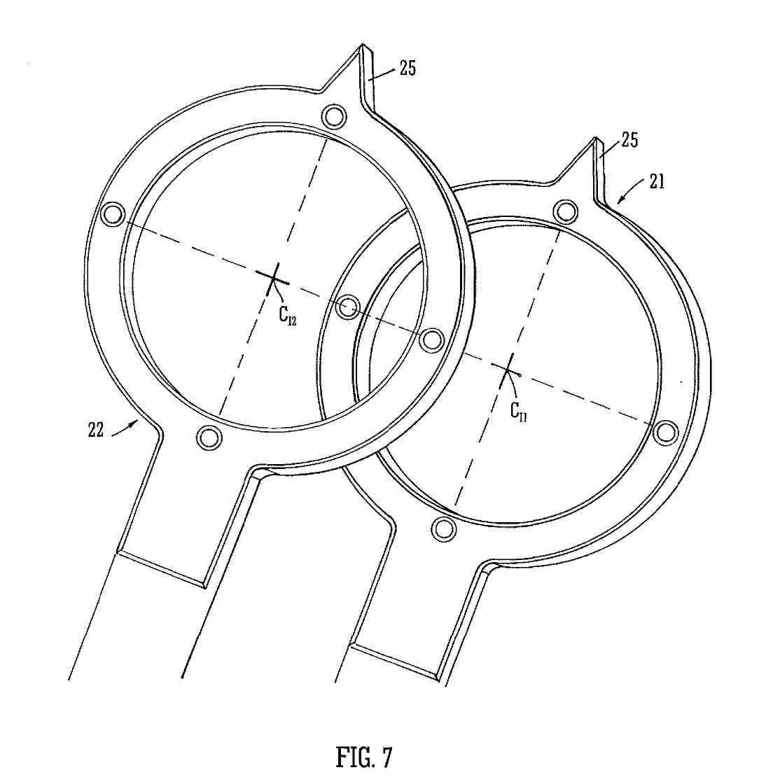

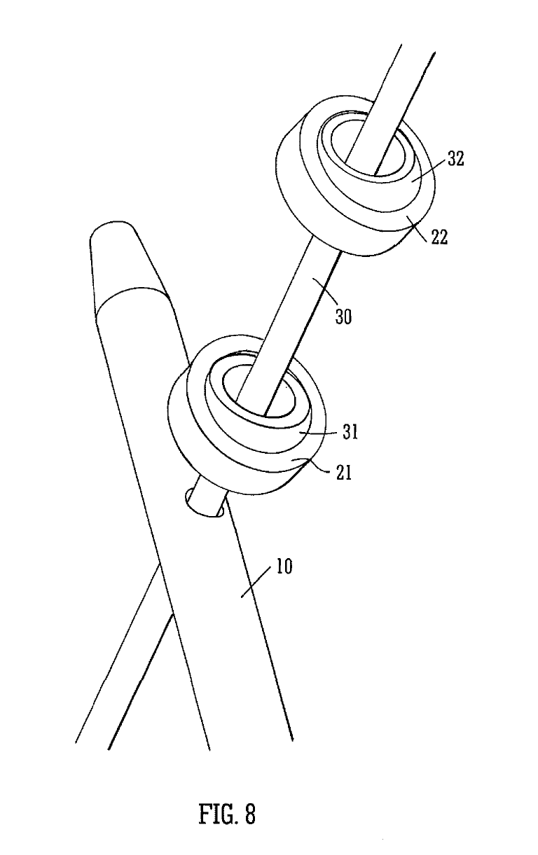

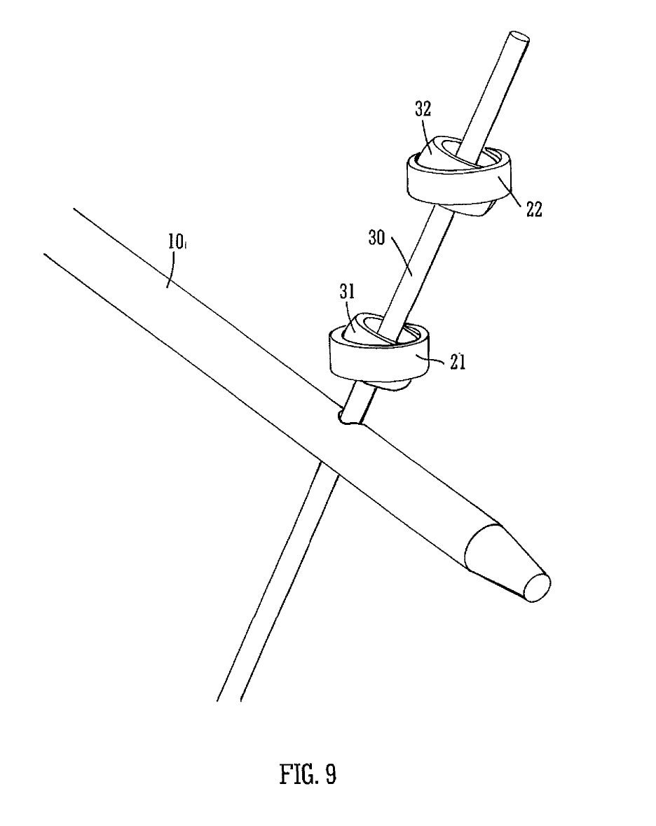

As shown in FIGS. 8-10, each of the indicators 21, 22 may be provided with a spherical ball 31, 32 located therein, each ball having a hole therethrough for receipt of the drill 30 or other tool. The spherical balls 31, 32 are moveable with respect to and within the indicators 21, 22 to provide a further degree of articulation to enable the centres (strictly of the spherical ball holes now, rather than the centres of the indicators 21, 22) to be aligned with one another and with the centre point C_{H} of the hole in the IM.

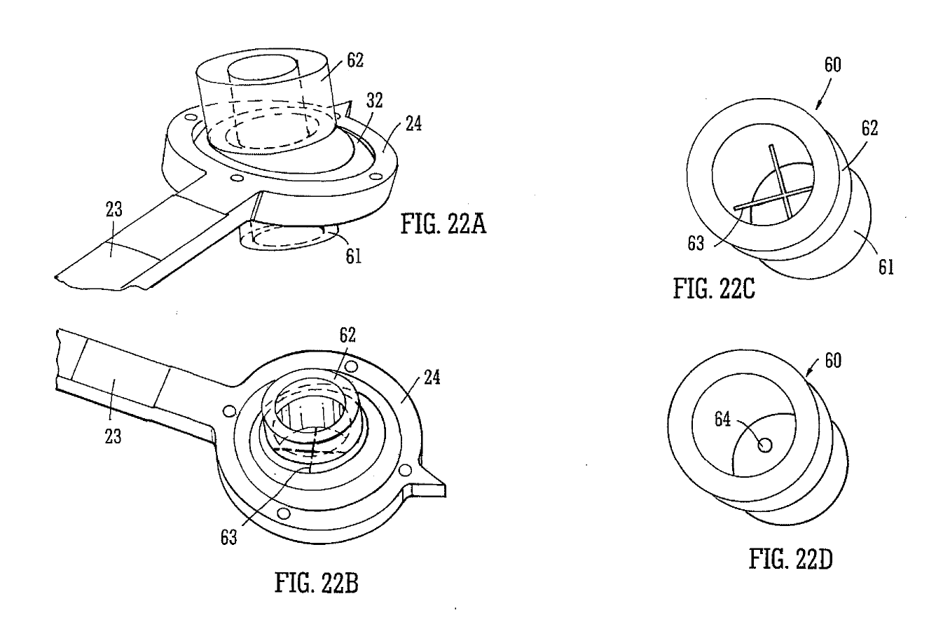

To further facilitate alignment of the centre of the indicators 21, 22, there may be provided an alignment plug 60 of the types illustrated in FIGS. 22A-22D.

The alignment plug 60 has a cylindrical shaft 61 of suitable diameter to fit snugly within the hole of the spherical ball 32 and an enlarged head 62 which prevents the plug falling through the hole. The plug 60 is made from a radiolucent material such as PEEK or any other suitable material.

Located within the plug 60, preferably within the shaft 61, is provided a radiopaque cross-hair 63 or other locator which can be used to facilitate location of the centres and C_{I2}. The cross hair is orientated substantially perpendicular to the longitudinal axis of the plug 60 and is preferably axially located such that, when the plug is placed in the spherical ball 32 (as shown in FIGS. 22A and 22B), the cross-hair is axially aligned with the centre of the spherical ball 32. In use, the cross-hair provides a readily visible locator to assist with aligning the centres of the spherical balls 31, 32.

The locator 63 need not be a cross-hair, for example a single radiopaque bead or ball 64 located in the shaft of the plug 60 may be used as illustrated in FIG. 22D. Other alternatives can be envisaged.

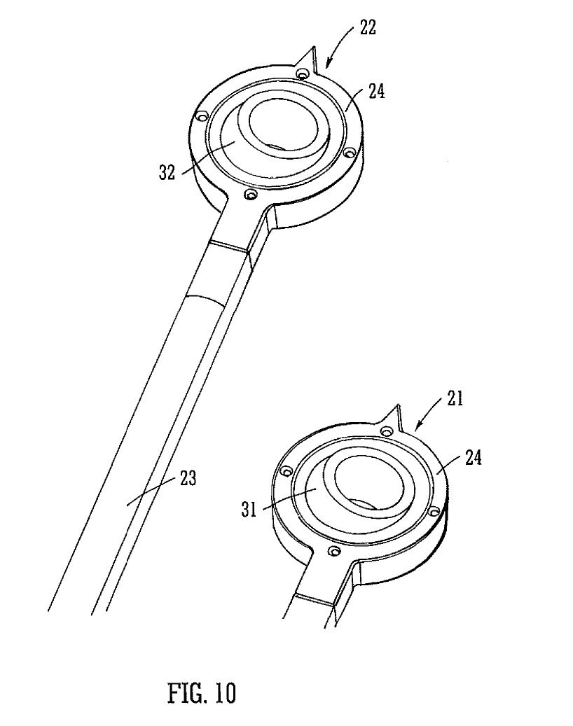

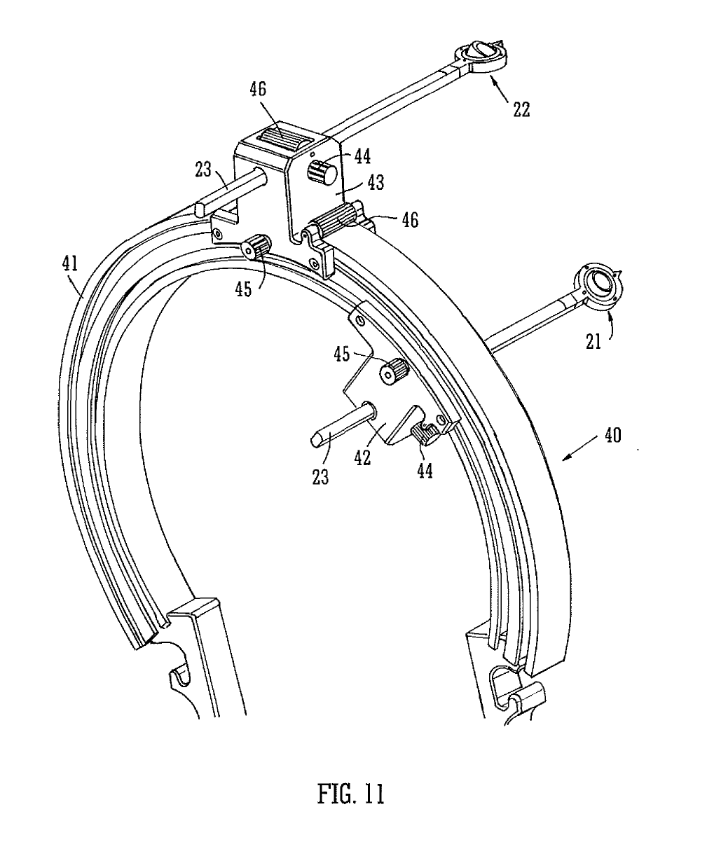

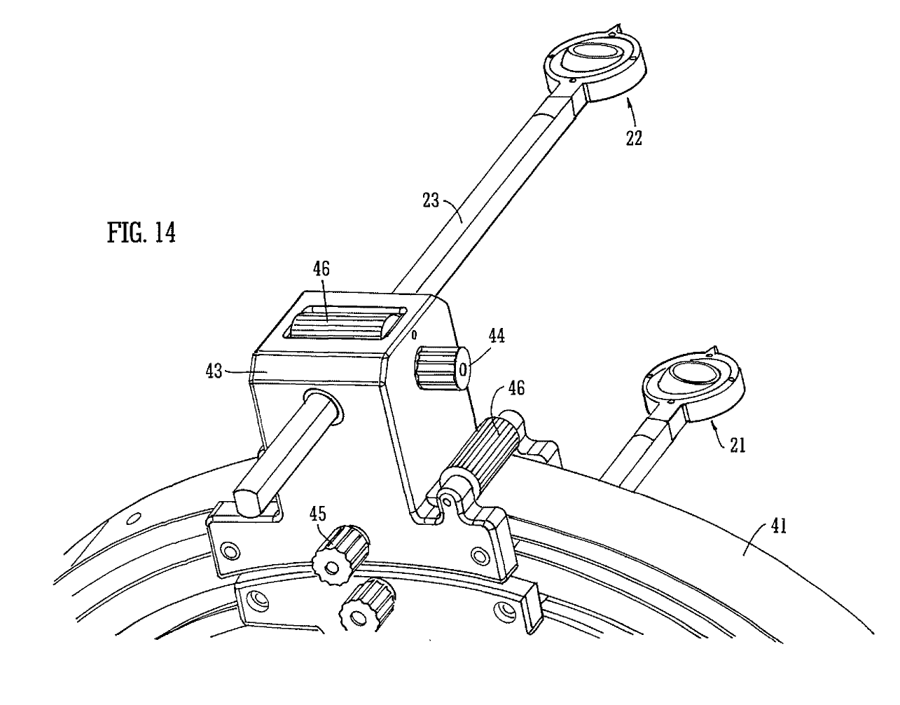

FIGS. 11-14 show the indicators 21, 22 mounted on a rig to enable alignment and locking in position with respect to the hole in the IM. The rig 40 comprises a rigid arc portion 41 and two moveable indicator mounts 42, 43, one for each of indicators 21, 22. The handle 23 of each indicator 21, 22 is located within an aperture in the respective indicator mount 42, 43 with each indicator 21, 22 being moveable along its own longitudinal axis in and out of its indicator mount 42, 43 so that the length of each handle projecting out of the working side of the indicator mount (the right hand side of FIG. 11) can be selectively adjusted and locked in place using mount lock screw 44.

Each of the indicator mounts 42, 43 is independently moveable in either direction around the arc portion 41 along a track and lockable in place using arc lock screws 45. Movement along the track may be achieved, for example, by smooth plastic spheres inside the indicator mount acting as rolling elements in the track on the arc portion 41.

Rollers 46 may be provided to facilitate fine adjusting movement of the indicator mounts and indicator handles therein.

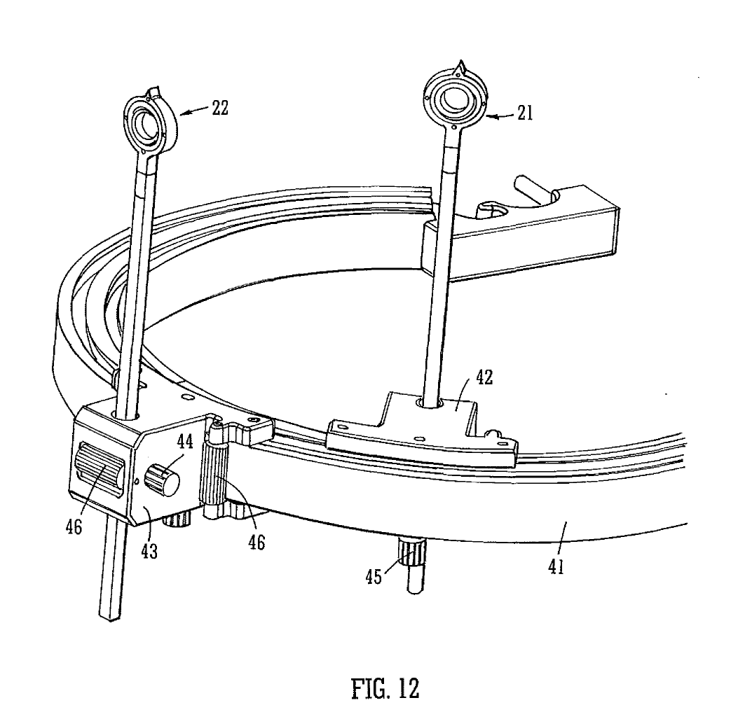

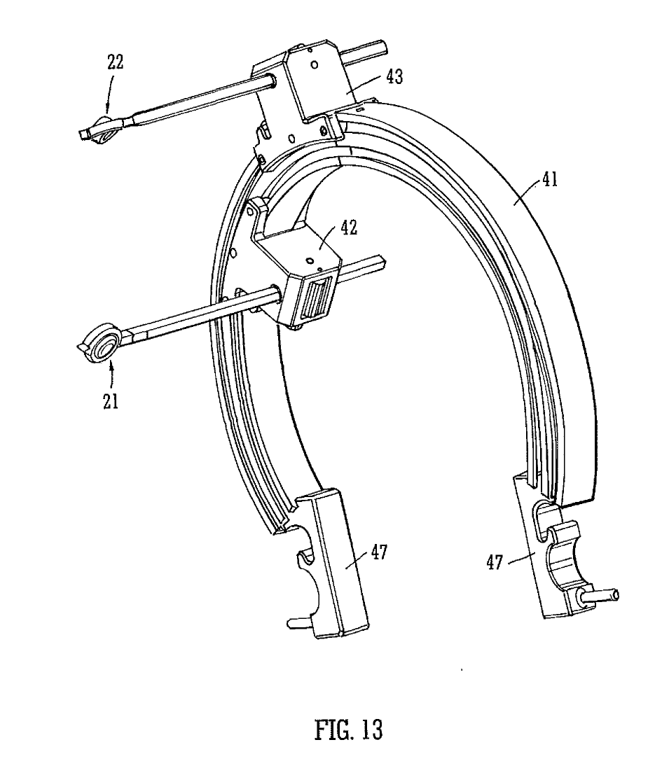

The arc portion 41 is so shaped so that it can fit generally around and in close relation to the distal end of the patient's leg in use, fixed rigidly to a frame or the like (not illustrated) so that the arc portion 41 cannot move. The arc portion could be located on the known STORM® fracture reduction machine. FIG. 13 for example shows suitable fixings 47 for attachment to a STORM® machine. Alternatively, the arc portion could be fixed to any suitable table, frame or the like. With reference to FIG. 12, the patient's leg and implanted IM would be oriented generally from the bottom to top of FIG. 12, passing through the middle of the arc portion 41. Ideally, indicator 21 is located as close as possible to the skin of the leg.

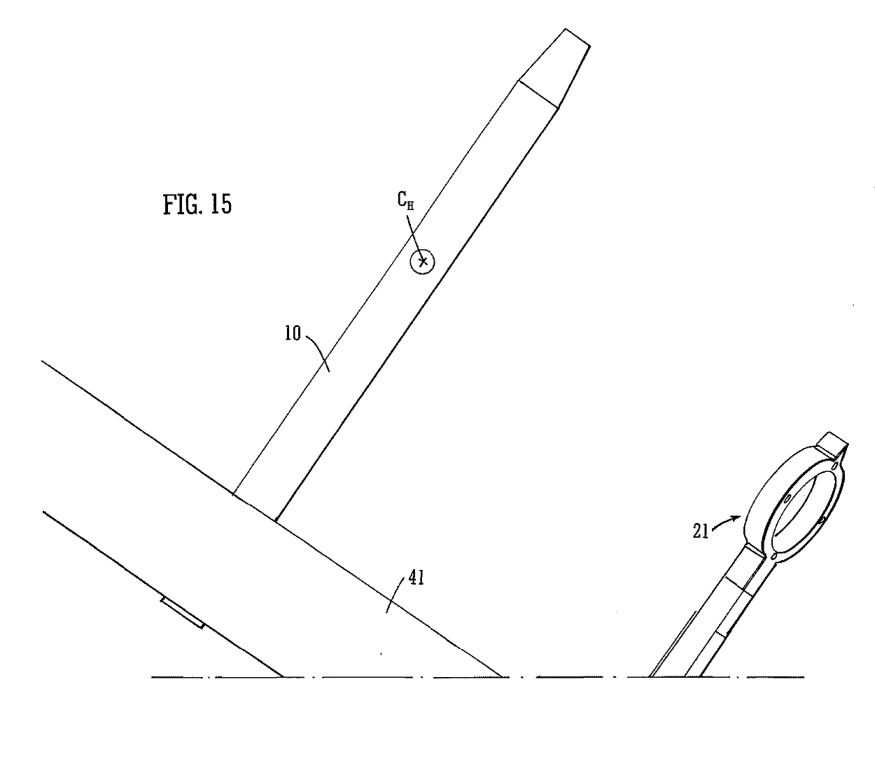

To use the apparatus, a radiographer initially locates the hole in the IM using known techniques, providing a view of the hole as illustrated in FIG. 15. The arc portion 41 of the rig is partially visible in FIG. 15 as is one of the indicators but it is to be noted that, at this initial stage, the indicators could be located at any position around the arc and with any length of handle protruding from their respective indicator mounts.

Next, the first of the indicators (preferably the indicator 21 nearest the leg) in its indicator mount is moved along the track around the arc portion 41 until it is roughly aligned with the centre point C_{H} of the hole in the IM. The indicator mount 42 is then locked with respect to the arc portion using lock screw 45.

Then, by releasing lock screw 44, the handle of the indicator 21 can be moved into or out of the indicator mount 42 so as to better align the centre point C_{I1} of the indicator 21 with the centre point C_{H} of the hole in the IM. The handle 23 is then locked with respect to the indicator mount 42 using lock screw 44 to prevent further movement along the longitudinal axis of the handle.

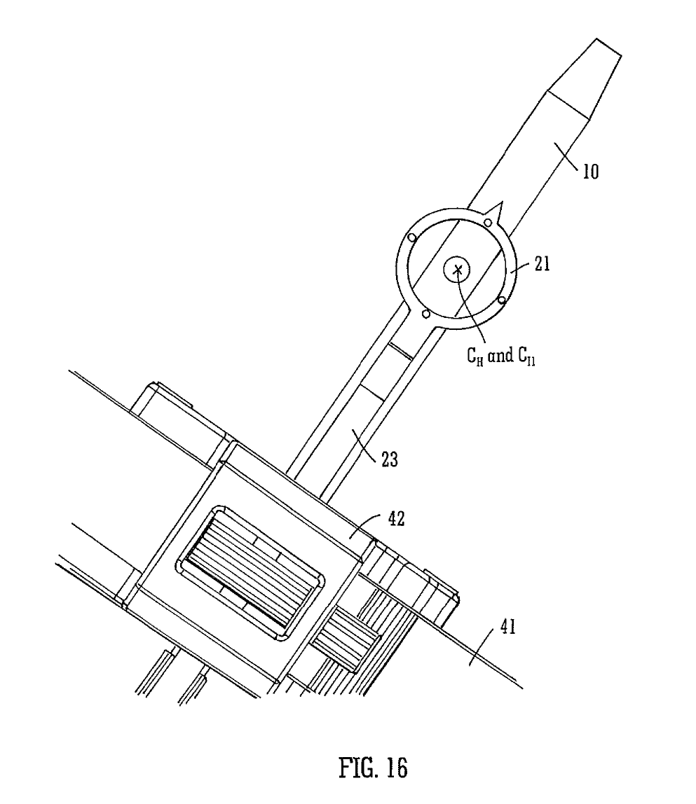

Further adjustments to the position of the centre point C_{I1} of the indicator 21 with respect to the centre point C_{H} of the hole in the IM can be made by selectively releasing either lock screw 45 (to permit movement of the indicator mount around the arc portion track) or lock screw 44 (to permit movement of the indicator handle along its longitudinal axis in or out of the indicator mount) until accurate alignment of C_{I1} and C_{H} is achieved as shown in FIG. 16.

Whilst one indicator identifies the location of the hole, it does not provide sufficient structural integrity to insert a drill (for example) into the hole. A second alignment device is required to produce a second centre and hence a line joining the two centres that is also in alignment with the axis of the hole. In the present invention, it is the axial alignment, and its maintenance during any further procedure, that is integral to successful detection and insertion.

Once the indicator 21 has been aligned with the centre point C_{H} of the hole in the

IM, the above process is repeated for the second indicator 22, resulting in all three centre points C_{H}, C_{I1} and C_{I2} being aligned, as shown in FIG. 5C. The indicators 21, 22, indicator mounts 42, 43 and arc portion 41 are all locked with respect to one another so that the alignment cannot be disrupted.

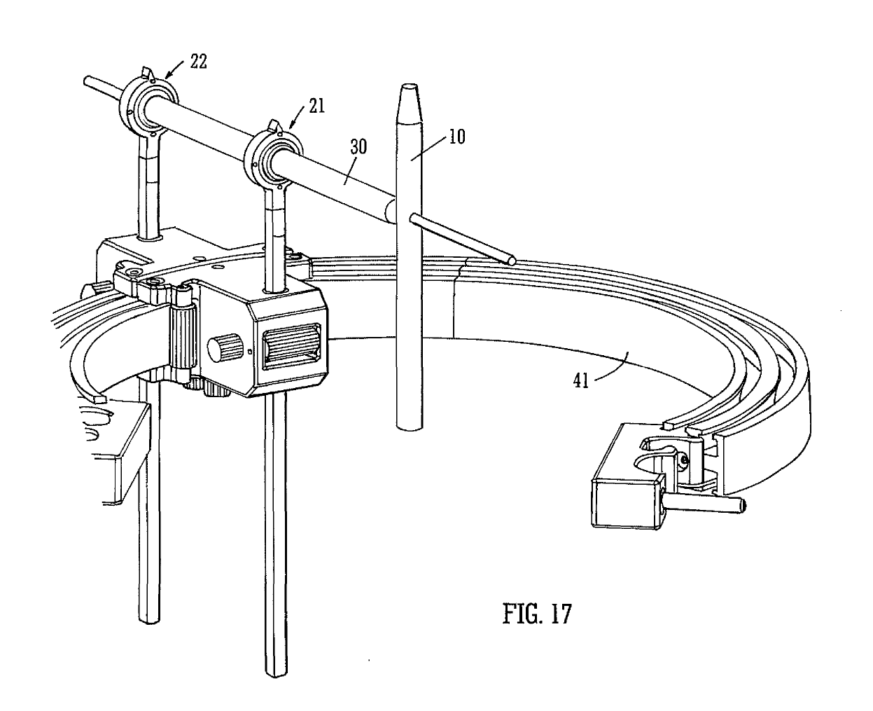

Once properly aligned, the holes through the indicators 21, 22 provide a direct guide for a drill or other tool into the hole in the IM. FIG. 17 shows a drill 30 in place.

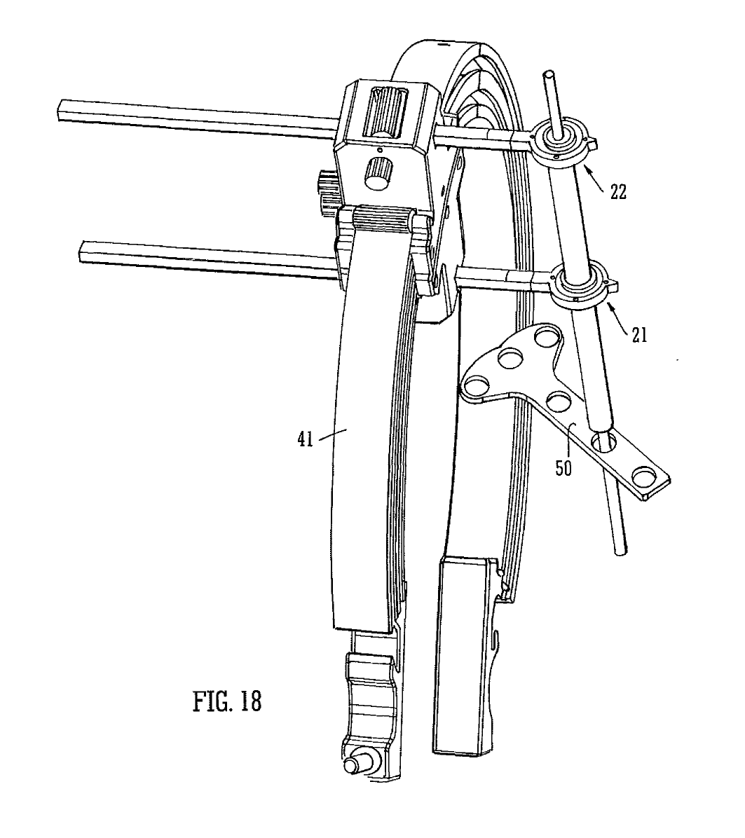

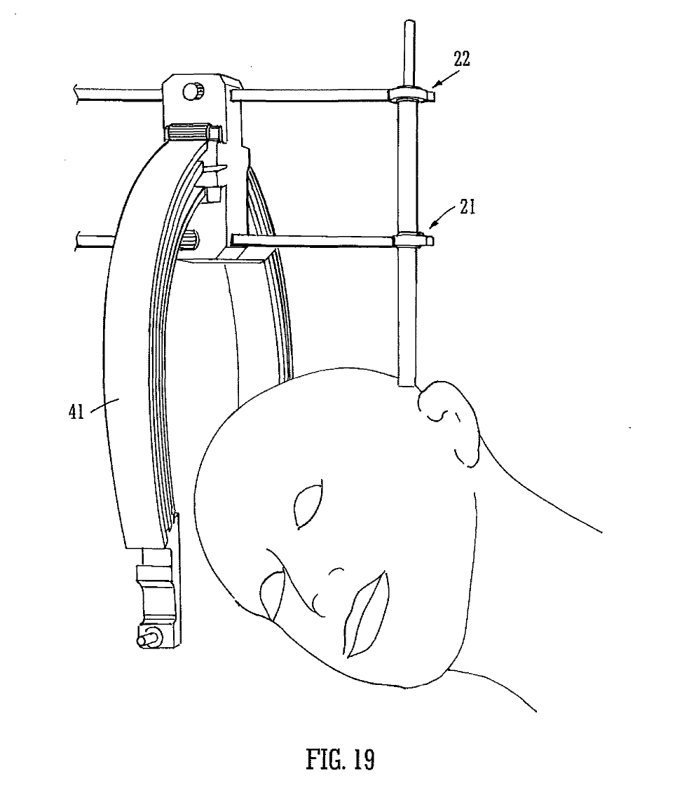

Although the above example has been described in relation to locating a target hole in an intramedullary nail, the invention is equally applicable to many other surgical applications in other parts of the body. The “target hole” of claim 1 could include any target site whose centre point is imageable so as to be identifiable e.g. using an X-ray monitor. For example, as shown in FIG. 18, the apparatus could be used to locate a hole in an implanted radiopaque bone plate 50 or, as shown in FIG. 19, the apparatus could be used for locating or positioning in any part of the body. The indicators are at least partially radiopaque to some part of the EM spectrum such that they are imageable so that their respective centre points can be identified. The indicators may be imaged by x-ray or infrared means, for example. Alternatively, the indicators may be imaged simply by the human eye, where a direct visual path is available between the viewer and the indicator.

Other imaging means may be used to image the indicators and identify their respective centre points within the scope of the present invention. These may avoid the time-consuming and repetitive need to position the apparatus, move personnel away to take an X-ray image, move personnel back to adjust the apparatus and so on. Examples of alternative imaging means include ultrasound, or having an emitter located in the region of the target hole (in an intramedullary nail for example) and a detector for detecting the position of the target hole as a reference point.

If the patient's limb is fixed in a jig such as the STORM® fracture reduction device, and one or more datum/reference points are available, stored rather than real-time images may be used. For example, one or more X-ray images or one or more CAT scan images could be used to determine relevant coordinates, angles and alignments. These images and/or the resultant data can be used to align the indicators.

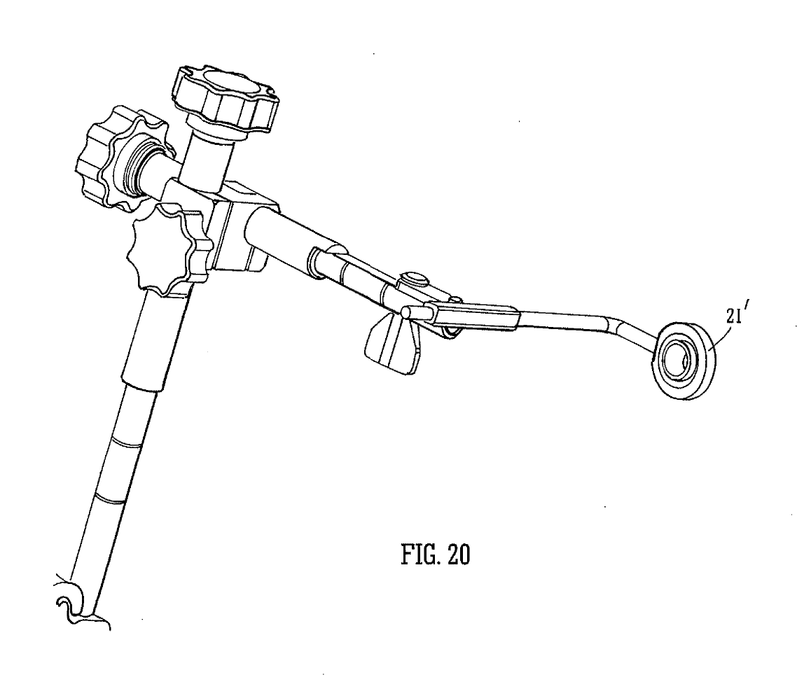

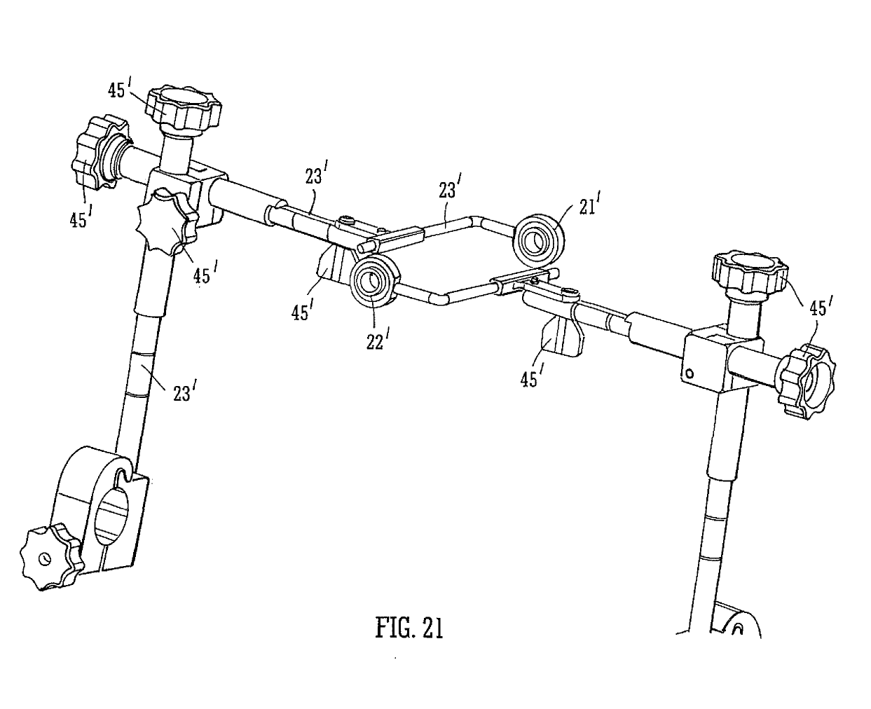

The embodiment utilising an arc portion 41 is described by way of example only. Alternative embodiments can be envisaged having different structures, but still using the same principles of pre-aligning an X-ray machine with the centre point of a target (e.g. hole in IM nail) and then aligning two independently moveable radiopaque indicators having identifiable centre points. FIGS. 20 and 21 show an alternative embodiment which provides freedom of movement to indicators 21′, 22′ using a combination of slidable and/or pivotable handles 23′ lockable with a plurality of lock screws 45′.

Since the methodology of the present invention relies on the alignment of two circles (or any suitable 2D/3D shape), the skilled reader will appreciate that various methods of achieving this effect are possible within the scope of the present invention. The embodiment of FIGS. 20 and 21 utilises the two slidable and/or pivotable handles 23′ (or “arms”) that allow rotational and translational movement of each individual indicator 21′, 22′, thus achieving a similar result to the previously described embodiment. The embodiment of FIGS. 20 and 21 is particularly preferable where the use of an arch would not be feasible or practical. Equally, the skilled reader will appreciate that further embodiments are within the scope of the present invention, where, for example, one or more computer controlled or robotic arms are used in place of the above-described manually-manipulated members.

Computer-controlled or robotic arms have the significant advantage of no longer having the surgeon's hands in the imaging field. Instead of the surgeon positioning the apparatus, having to move away to take an X-ray image, then moving back to adjust the apparatus etc, the imaging can be done continuously and in real-time, with the image presented to the surgeon safely away from the imaging field, the surgeon controlling automated adjustment of the apparatus remotely.

Automated or motorised (rather than manual) control of the individual indicators 21, 22 can be achieved by incorporating motors into the indicator mounts 42, 43. Alternatively, and to avoid the need for the motors to be sterile, the motors could be located remotely with sterile cable drives into the indicator mounts.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A targeting guide for locating a surgical target site, for example a hole in a distal end of an implanted intramedullary nail, the targeting guide comprising:
a first indicator having an identifiable reference point, the first indicator being mounted on a first handle having a first longitudinal axis;
a second indicator having an identifiable reference point, the second indicator being mounted on a second handle having a second longitudinal axis;
the indicators being radially spaced from one another with respect to said target site and being at least partially radiopaque so that they are imageable such that their respective reference points can be identified;
a rig on which the first and second indicators are mountable such that each indicator is independently moveable with respect to the rig along the respective longitudinal axis of its handle, independently moveable with respect to the rig along at least one further axis and lockable with respect to the rig in a selected position along each of said axes;
wherein, in use, the reference point of each indicator is capable of being aligned with a reference point at the target site using imaging means, the aligned indicators providing a guide to the target site for a drill, trocar or other surgical instrument or tool.

2. The targeting guide of claim 1 wherein the identifiable reference point of said first indicator and/or said second indicator and/or said target site is a centre point.

3. The targeting guide of claim 1 or claim 2 wherein said rig comprises an arc portion and movement along said at least one further axis comprises movement along said arc portion.

4. The targeting guide of claim 1 wherein movement along said at least further axis comprises a pivoting movement.

5. The targeting guide of claim 1, wherein said handles are radiolucent.

6. The targeting guide of claim 1, wherein said first indicator is a ring.

7. The targeting guide of claim 6 wherein the ring has one or more alignment markers thereon to facilitate location of said identifiable reference point.

8. The targeting guide of claim 7 wherein at least one of said alignment markers is a pointer extending radially with respect to the centre point of said ring.

9. The targeting guide of claim 7 wherein at least one of said alignment markers is a radiolucent hole through a peripheral surface of said ring.

10. The targeting guide of claim 1 wherein the first indicator and second indicators are the same shape.

11. The targeting guide of claim 1, wherein said rig portion includes fixings for attachment of the rig portion to a fracture reduction machine, table, frame or other supporting structure.

12. The targeting guide of claim 1, wherein the rig portion comprises a two-part rig portion, the first indicator being mountable on a first part of the rig portion and the second indicator being mountable on a second part of the rig portion.

13. The targeting guide of claim 1, wherein the rig portion comprises at least one mounting block for at least one of said first and second indicators.

14. The targeting guide of claim 13 wherein the rig portion comprises first and second mounting blocks for said first and second indicators respectively.

15. The targeting guide of claim 13 wherein the or each mounting block is moveable with respect to said rig portion, for example by running the mounting block along a track on said rig portion.

16. The targeting guide of claim 15 wherein the or each mounting block is moveable with respect to said rig portion by means of remotely located or locally positioned motor means.

17. The targeting guide of claim 1, wherein said movement of said first and second indicators can be finely adjusted using controls locally positioned, for example on said mounting blocks, or located remotely.

18. The targeting guide of claim 1, wherein said rig further comprises a locking mechanism for independently selectively locking each of said first and second indicators in a desired position.

19. The targeting guide of claim 1, wherein said first and/or said second indicators include a generally spherical bearing therein, a reference point of said bearing being generally aligned with the reference point of the indicator, the bearing being able to articulate with respect to the indicator and the bearing having a hole therethrough for receipt of a drill, trocar or other surgical instrument or tool.

20. The targeting guide of claim 19 wherein said spherical bearings comprise plastics or any other radiolucent material.

21. The targeting guide of claim 1, further comprising a radiolucent plug containing a radiopaque cross-hair or other radiopaque indicator, the plug being suitably sized and shaped to fit within the bore of said indicators and/or said spherical bearings.

22. The targeting guide of claim 21 wherein said plug has a generally cylindrical shaft and an enlarged head.

23. The targeting guide of claim 1, wherein said indicators comprise metal.

24. The targeting guide of claim 1, wherein said first indicator is radially nearer said target site than said second indicator.

25. The targeting guide of claim 1, wherein the indicators are at least sufficiently radiopaque so that they cast an image on an X-ray monitor from which their respective reference points can be identified, and wherein the imaging means comprise an X-ray monitor.

26. The targeting guide of claim 1, further comprising a computerised or robotic control system for moving the first and second indicators with respect to the rig.

27. The targeting guide of claim 1, wherein the surgical target site is a hole in a distal end of an implanted intramedullary nail.

28. A method of locating a surgical target site using a targeting guide of claim 1, comprising the steps of:
mounting the rig of said targeting guide in the vicinity of said target site;
aligning an imaging means with the reference point of the target site;
moving said first indicator along said first longitudinal axis and/or along said further axis until its reference point is aligned with the reference point of the target site;
locking said first indicator with respect to said rig;
moving said second indicator along said second longitudinal axis and/or along said further axis until its reference point is aligned with the reference point of the target site;
locking said second indicator with respect to said rig.

29. The method of claim 28, wherein a computerised or robotic control system is used to at least partially move the first and second indicators with respect to the rig.

30. A method of locating a surgical target site using a targeting guide of claim 1.
